(19) United States
(12) Patent Application Publication
(10) Pub. No.: US 2018/0207381 A1
(43) Pub. Date: Jul. 26, 2018

(54) Tracheostomy tube with removable neck flange

(71) Applicant: Winthrop Innovations, LLC, West Palm Beach, FL (US)

(72) Inventors: Neil Winthrop, West Palm Beach, FL (US); Kimberly Winthrop, West Palm Beach, FL (US)

(21) Appl. No.: 15/873,689

(22) Filed: Jan. 17, 2018

Related U.S. Application Data
(60) Provisional application No. 62/448,735, filed on Jan. 20, 2017.

(51) Int. Cl. A61M 16/04

(52) U.S. Cl. CPC A61M 16/0465; A61M 16/0475; A61M 16/0427; A61M 16/0488

(57) ABSTRACT

A tracheal tube assembly includes an outer cannula configured to be positioned in a patient airway and an inner cannula configured to be disposed inside the outer cannula. The tracheal tube assembly includes a removable neck flange bracket with trach tie adapter points secured about the outer cannula, and a connector coupled to a proximal end of the outer cannula. The connector can include a swivel design feature and a flexible tubing connector may be formed with the connecter. The connector is constructed and arranged to provide a view of the inner cannula, and the inner cannula and the connector form a contiguous passageway for exchanging fluid with the patient airway in operation.

### PRIORITY APPLICATION

In accordance with 37 C.F.R. § 1.76, a claim of priority is included in an Application Data Sheet filed concurrently herewith. Accordingly, the present invention claims priority to U.S. Provisional Patent Application No. 62/448,735, entitled “TRACHEOSTOMY TUBE WITH REMOVABLE NECK FLANGE”, filed Jan. 20, 2017. The contents of the above referenced application is incorporated herein by reference.

### FIELD OF THE INVENTION

The present disclosure relates generally to the field of tracheal tubes and, more particularly, to a tracheostomy tube having a rotatable and removable neck flange.

### BACKGROUND OF THE INVENTION

Artificial ventilation of a patient may be desired during certain surgical procedures or in treatment of certain conditions. Typically, an endotracheal tube may be inserted through the mouth to provide oxygen and other gasses to a patient. When long term intubation is necessary, a tracheostomy tube is preferred. Tracheostomy tubes are typically inserted through an incision made through the trachea. A resulting stoma is formed between the tracheal rings below the vocal chords. The tracheostomy tube is then inserted through the opening.

A tracheostomy tube generally refers to a tube or catheter inserted into the trachea of a patient for the purpose of establishing and maintaining an open airway for the patient. The tracheostomy tube is inserted into a tracheotomy stoma in order to permit the patient to breathe without the use of their nose or mouth. The tracheostomy tube includes a relatively short (two to four inches) curved tube or cannula with a balloon cuff located at a distal end. The balloon cuff is inserted into the patient's trachea and serves to open the patient's airway. It is noteworthy that some tracheostomy tubes may not include a cuff. The tracheostomy tube additionally includes a connector/adapter and a neck plate located near the proximal end. The connector adapter is configured for connection with additional hosing, e.g., connected to an oxygen source. The neck plate rests against the patient's neck and serves as a securing mechanism for the tracheostomy tube. The neck plate generally includes elongated holes on opposing ends, lateral with respect to the stoma. Trach ties (e.g., elastic or fixed straps with Velcro′ on one or each end) may be wrapped around the back of the patient's neck, inserted through the elongated holes on the neck plate, and secured using the Velcro′ so as to secure the tracheostomy tube.

Conventionally, tracheostomy tubes include an inner cannula positioned inside the tracheostomy tube and used as a conduit for liquids or gas incoming and outgoing into a patient's lungs. The inner cannula may be removed for cleaning or disposal without disturbing the placement of the tracheostomy tube. A connector is typically provided at an upper or proximal end where the tube exits the patient airway. The connector may be attached to a ventilator or the like device. The neck flange on most trach tubes is rigid and usually fixed in place. Most of them will articulate mildly upward down for patient comfort.

Standard connectors have been developed to allow the tracheal tube to be fluidly coupled to artificial ventilation equipment to supply the desired air or gas mixture to the patient, and to evacuate gases from the lungs. One difficulty that arises in the use of tracheal tubes, and tracheostomy tubes in particular, is in the connection of the tube to the ventilation equipment. For example, an inner cannula may not be installed, or may be installed improperly. This may lead to difficulties with ventilation when a connection is made to ventilation equipment

U.S. Pat. No. 3,569,612 is has an inner and an outer cannula placed within the trachea of a patient. The inner cannula may be connected to a respirator if respiratory assistance is required. If the inner cannula is obstructed, it can be removed from the outer cannula and the obstruction thereafter extracted from the inner cannula. During the time the inner cannula is removed, the patient breathes through the outer cannula. No provision is made for attaching a respiratory to the outer cannula so that the patient may receive respiratory assistance while the inner cannula is removed.

U.S. Pat. No. 9,511,201 discloses a tracheal tube having a viewable inner cannula. A tracheal tube assembly includes an outer cannula configure to be positioned in a patient airway and an inner cannula configured to be place inside the outer cannula.

U.S. Pat. No. 9,480,808 discloses a tracheostomy devices with a substrate having an opening that is disposed centrally on the substrate, the opening being configured to receive a connector of a tracheostomy tube, the substrate comprising a plurality of mounting holes disposed on opposing ends of the substrate, the substrate having a slit that extends below the opening which allows the substrate to be removed from the connector when the connector is connected to an airway hose.

What is lacking in the industry is a tracheostomy tube with a removable net flange that allows cleaning outside the trach tube and improves comfort and range of motion by use of a swivel design and a flexible tubing connector.

### SUMMARY OF THE INVENTION

Disclosed is a tracheal tube assembly that includes an outer cannula configured to be positioned in a patient airway and an inner cannula configured to be disposed inside the outer cannula. The tracheal tube assembly includes a removable neck flange bracket with trach tie adapter points secured about the outer cannula, and a connector coupled to a proximal end of the outer cannula. The connector can include a swivel design feature and a flexible tubing connector may be formed with the connecter. The connector is constructed and arranged to provide a view of the inner cannula, and the inner cannula and the connector form a contiguous passageway for exchanging fluid with the patient airway in operation.

An objective of the invention is to disclose an improved tracheal tube, and particularly for improved tracheostomy tubes, that allows for more comfortable trach tube ties changes, trach care including stoma cleaning and gauze changes, and less pressure exerts on the patients airway when connected to a ventilator or other oxygen delivery devices.

Another objective of the invention is to disclose an improved tracheal tube having a swivel design feature as well as a flexible tubing connector.

Another objective of the invention is to provide a tracheostomy tube that may be provided as a stand-alone device o be included in a kit that includes, but is not limited to: gloves for use by a caretaker in handling said kit, a tray for holding of fluids, a container for storing hydrogen peroxide, said hydrogen peroxide placed in a first compartment of the tray for used cleaning the tracheostomy support device and neck area of an individual, a cotton swap for use in cleaning the tracheostomy support device and neck area of an individual, a brush for use in cleaning the tracheostomy support device and neck area of an individual, a pipe head for use in cleaning the tracheostomy support device and the neck area of an individual, a container of sterile water for rinsing said tracheostomy neck flange and the neck area of the individual a gauze sized for placement between the tracheostomy neck flange and the neck area of the individual, and a plurality of ties for securing said neck flange to an individual.

An advantage of the instant invention is that the outer cannula is the trach tube that goes into the patient's airway/neck. The inner cannula can be fenestrated as well as non-fenestrated. In this disclosure, fenestrated indicated a means to conduct air from the tube to the patient's mouth.

Other objectives and further advantages and benefits associated with this invention will be apparent to those skilled in the art from the description, examples and claims which follow.

### BRIEF DESCRIPTION OF THE DRAWINGS

FIG. 1 is a perspective view of the instant invention;

FIG. 2 is a rear view of FIG. 1;

FIG. 3 is an enlarged front view;

FIG. 4 is a side view;

FIG. 5 is a perspective view of an inner cannula;

FIG. 6 is an exploded view of a cannula connector and neck flange; and

FIG. 7 is a top plane view of an alternative embodiment of the neck flange.

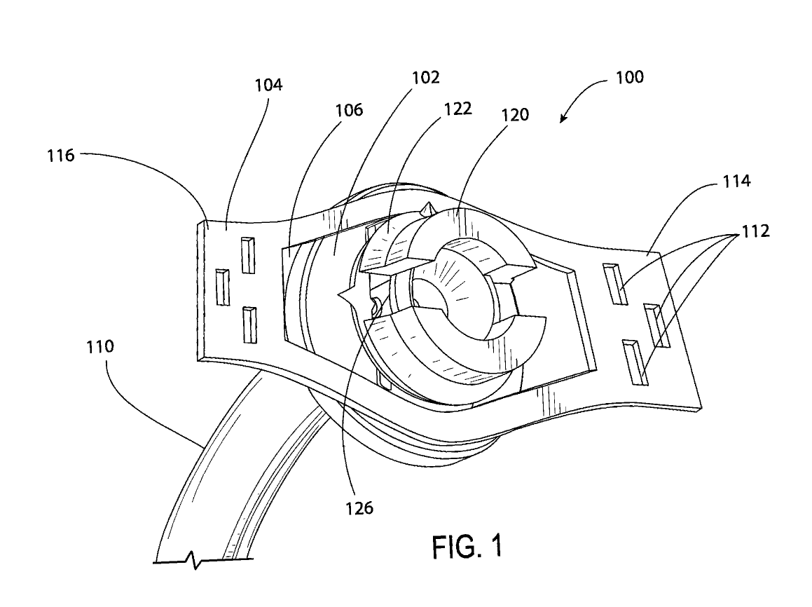

### DESCRIPTION OF THE INVENTION

Detailed embodiments of the instant invention are disclosed herein, however, is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

The space between the bracket and the collar is where a flexible or a non flexible removable neck flange/trach tube holder attachment is located. Not shown are tracheostomy tube ties for securing to the neck of an individual. This device may be inserted in the space between the bracket and the collar and can spin 360 degrees allowing full range of motion and improved patient comfort. The device can be clear plastic or solid in color, flexible or non flexible. The tracheostomy tube may be magnetic resonance imaging (MRI) compatible, thus allowing for artificial ventilation during MRI procedures.

Referring to Figure in general, illustrated is an exemplary tracheostomy tube holder 100 constructed for securement to a patient's neck. The holder 100 is formed from a body flange 102 constructed and arranged for releasable securement to a neck flange 104 using opening 106 which allows for placement in a space 105 beneath a cuff 122. The neck flange 104 is used to secure the tracheostomy tube 110 to the patient using, for example, trach ties. The neck flange 104 having tie mounting holes 112 located along a first end 114 and a second end 116. At least a portion of the neck flange 104 is sized/shaped so as to correspond to, and align with the mounted holes. The tube assembly includes an outer cannula configured to be positioned in a patient airway and an inner cannula configured to be disposed inside the outer cannula. The flange member is secured about the outer cannula, and a connector coupled to a proximal end of the outer cannula. The connector is configured to provide a view of the inner cannula, and the inner cannula and the connector form a contiguous passageway for exchanging ventilator gases, atmospheric gases, or fluid with the patient airway in operation.

The tracheostomy tube 110 includes a connector 120 having a cuff 122. The cuff 122 forming a lip to engage an edge of the opening 106. The opening 106 preferably includes a translucent material allowing for ease of installation by allowing a clear view of the body flange during installation. The neck flange 104 is removable by pulling the neck flange 104 or rotating a quarter turn along spacing 126 which forms a locking system using a tab 103 that can only pass through the opening 106 when aligned with a notch 105. The neck flange 104 can be cleaned and reused, or replaced with a new neck flange allowing a soiled neck flange to be disposed of. The caretaker can then place, or replace, absorbent gauze (not shown) around the trach tube and reinsert the connector into the neck flange 104 providing clean installation as part of the tracheostomy care. This attachment allows for less trauma to the patients neck and trachea and permits ease of work for the care giver while doing tracheostomy care.

The mounting holes 112 can be most any shape and configuration. The mounting holes 112 are positioned along the ends 114 and 116, and can be rotated in respect to the tube 110 providing a variety of mounting options for the user. The user may select the mounting holes 112 on opposing sides of the holder flange 104 depending on the desired position and/or orientation of the tube 110 yielding an optimally unobstructed airway. The user may use a trach tie, not shown, and then secure the trach tie using Velcro, for example, so as to secure the tracheostomy tube to the patient. The connector may then be connected to an airway hose that is in turn connected to an oxygen system (not shown). One of ordinary skill in the art would be familiar with the use of such devices and the addition of the tubes and oxygen system would distract from the clarity of the drawings.

At least a portion of the neck flange 104 may be formed using a pliable material that is non-irritating or otherwise will not harm the skin of the patient. One such exemplary material may be silk tape. Certain aspects may provide for a first side to include a cushioned material so as to reduce abrasions and/or provide a more comfortable fit for the tracheostomy tube against the patient's skin. In one example, the first side may include a hydrocolloid material. Other aspects may provide for the second side to also include a cushioned material, e.g., the hydrocolloid material.

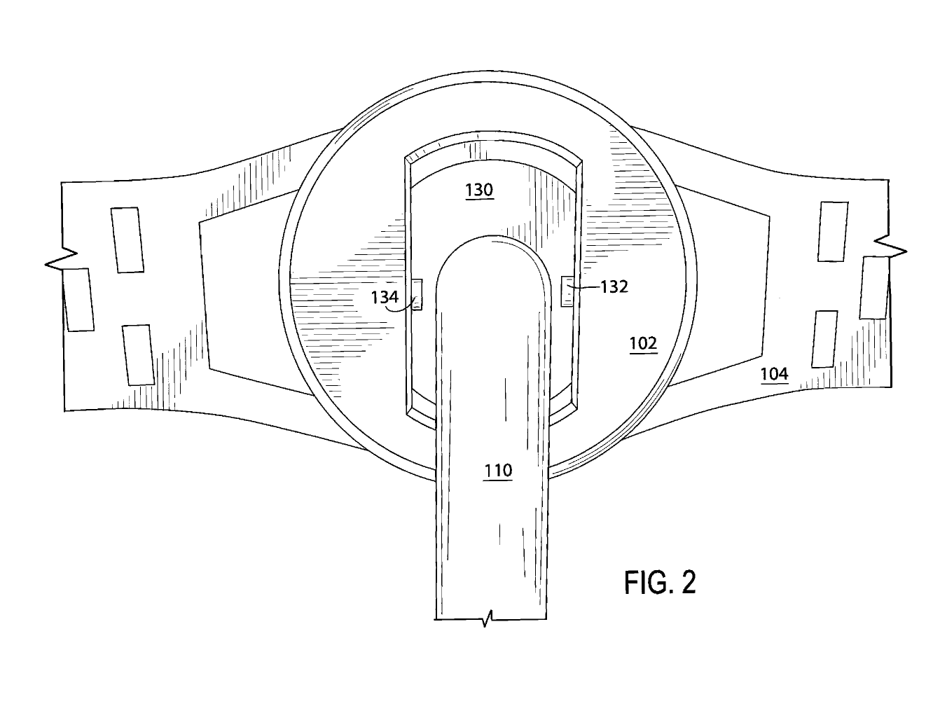

FIG. 2 is a rear view of the device 100 depicting the neck flange 104 overlying the body flange 102 and tracheostomy tube 110. The tracheostomy tube coupling 130 is illustrated with pivot pins 132 and 134 securing the coupling 130 to the body flange 102. The coupling allows for a comfortable attachment to the patient's neck.

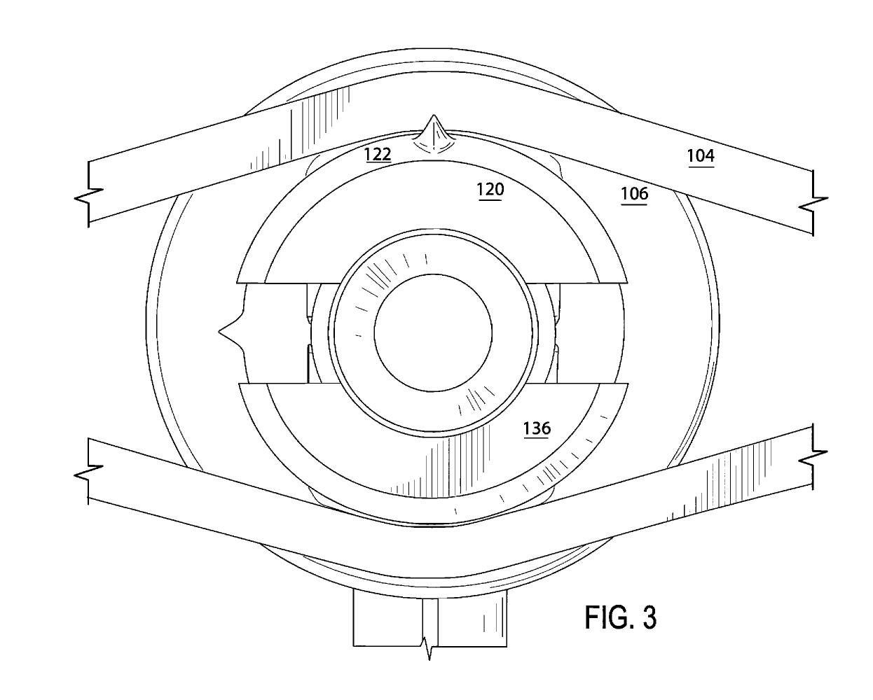

FIG. 3 is an enlarged front view of the device 100 illustrating the connector 120 having cuff 122. The cuff 122 forming a lip to engage an edge of the opening 106. The connector 120 provides a configuration as a trach holder attachment. The opening 106 preferably includes a translucent bordering material allowing for ease of installation by allowing a clear view of the body flange during installation.

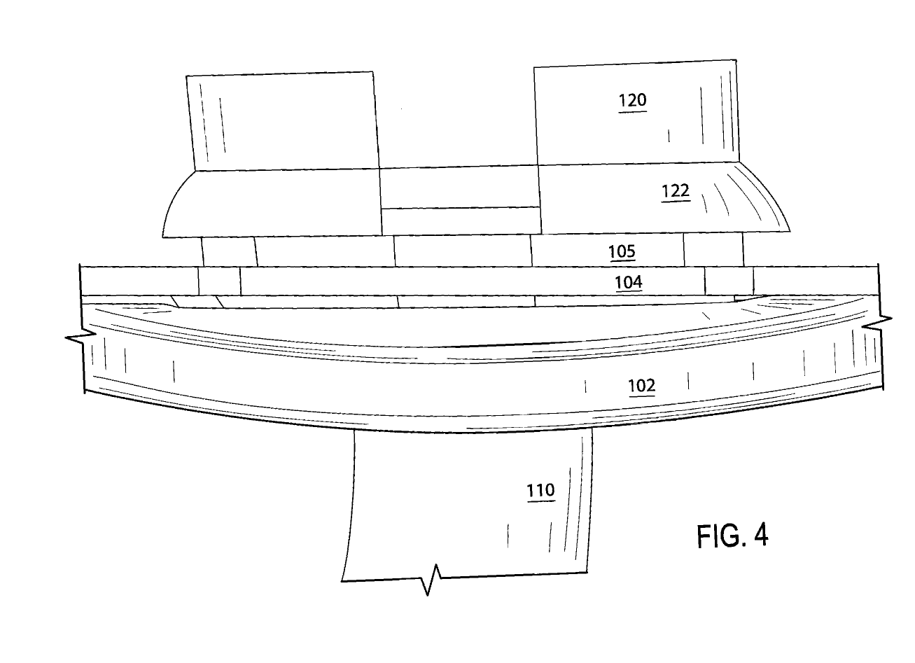

FIG. 4 is a side view of the device 100 illustrating the placement of the neck flange 104 positioned between the body flange 102 and cuff 122. The connector 120 providing rotation of a trach tube through a 360 degree rotation.

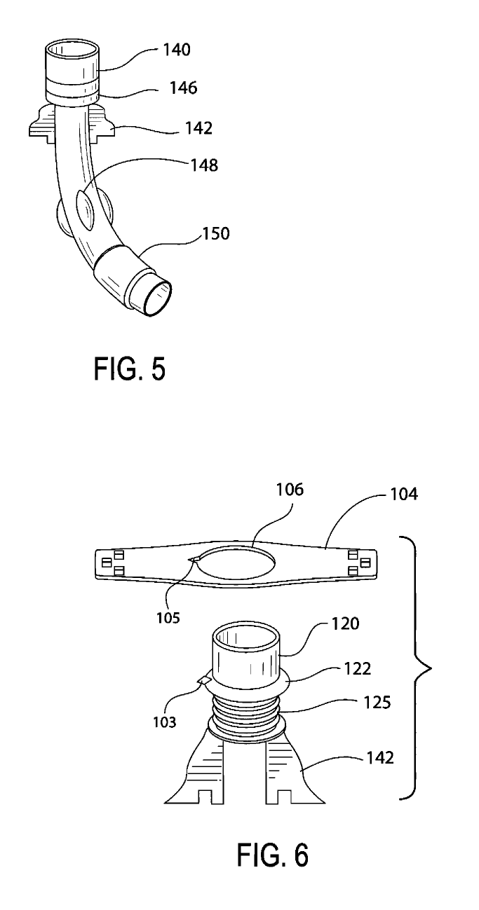

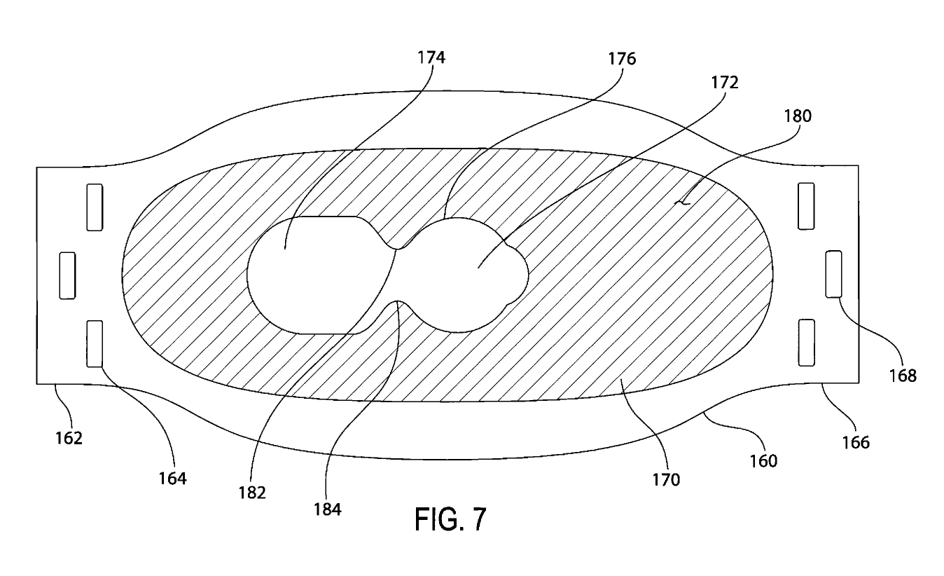

FIG. 5 is a view of an inner cannula 140 having connector tabs 142 for engagement. The connector tabs 142 securing to the spacing 126 area on the connector 120. The connector tabs 142 providing a positive lock to the cannula, the lock can be overcome by folding back the connector tabs to cause release.

Preferably these are 15 millimeter connectors for use on inner cannulas. The inner cannula 140 locks into place to the outer cannula of the tracheostomy tube. Adding a swivel design feature 146 allows a level on comfort to the patient, especially if the connecter has downward force placed on it from the ventilator circuit wherein the swivel will relieve lateral pressure exerted in the tracheal walls. The inner cannula can be fenestrated 148 and inflatable balloon 150 can be attached to the tubing.

In the preferred embodiment, the flexible tubing connector can be built right into the 15 millimeter connecter. The flexible tubing connector added to the 15 millimeter connecter will also improve the pressure exerted by the weight of ventilator tubing. The 15 millimeter connecter is where the patient will breathe through spontaneously and also where the ventilator would attach as well if they are connected to a ventilator. The flexible tubing assimilates a bendable straw having a plurality of peaks and valleys 125 that allow bending of the tube in any direction. As previously mentioned, the cannula may include an inflatable cuff or balloon for sealing of the cannula.

FIG. 5 depicts the connector 120 with the cuff 122 for engaging the opening of the neck flange 104. The removal of the neck flange 104 allows for ease of dressing changes, as well as installation. The neck flange 104 removal is performed by pressing the ends 114 and 116 causing the flange 104 to counter flex which releases the flange 104 from the cuff 122. In an embodiment the connector 120 has a swivel 125 to allow ease of rotation.

In an alternative embodiment, a neck flange 160 has end 162 with apertures 164 to receive ties, not shown. End 166 having apertures 168 to also receive ties. A central panel 170 may be formed of translucent material or opaque flexible material. The central panel 170 having an opening 172 with an insertion end 174 for, receipt of the tracheostomy tube having the cuff described above. The edge 176 of the central panel 170 fitting within the space 105 between the neck flange 104 and the cuff 122 illustrated in FIG. 4. The tube is placed into the insertion end 174 until the cuff 122 engages the upper surface 180 of the control panel 170. Pinch points 182 and 184 are flexible and expand momentarily as the cuff 122 is slide in position from the insertion end 174 to the opening 172. The pinch points 182 and 184 then return to the illustrated position in a manner similar to a cotter pin. When the cuff 122 is secured into the opening 176 the neck flange 160 can be used to secure the tracheostomy tube 110 to the patient in accordance with the previous disclosure.

The tracheostomy support device can be formed as part of a tracheostomy kit. In this embodiment the kit would comprise a tracheostomy support device having a neck flange having two ends with a centrally disposed opening therebetween, said neck flange releasable secured to a patients neck; a body flange releasably securable to said neck flange; and a tracheostomy tube having a connector with a cuff, said connector insertable into said body flange and said cuff having a lip to engage an edge of said opening to couple said tracheostomy tube to said neck flange; gloves for use by a caretaker in handling said kit; a tray for holding of fluids; a container for storing hydrogen peroxide, said hydrogen peroxide placed in a first compartment of the tray for used cleaning said tracheostomy support device and neck area of an individual; at least one cotton swap for use in cleaning said tracheostomy support device and neck area of an individual; at least one brush for use in cleaning said tracheostomy support device and neck area of an individual; at least one pipe head for use in cleaning said tracheostomy support device and neck area of an individual; a container of sterile water, said sterile water for rinsing said tracheostomy neck flange and the neck area of the individual; a gauze sized for placement between said tracheostomy neck flange and the neck area of the individual; and a plurality of ties for securing said neck flange to an individual.

It is to be understood that any of the herein described holders may be sized and/or shaped so as to correspond to the size or shape of the tracheostomy tube being utilized therewith. That is, tracheal tubes may be sized depending on the age or size of the patient, e.g., neo-natal, infant, child, young adult, adult, obese adult, etc. The size of the tracheal tube would determine the size of the components forming the components of the tracheal tube, e.g., the plate size, tube diameter, tube length, etc. The herein described holders may be sized so as to correspond to the size of the tracheostomy tube it is being used with. The above description is illustrative and not restrictive.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A tracheostomy support device, comprising:
a neck flange having two ends with a centrally disposed opening therebetween, said neck flange releasable secured to a patients neck;
a body flange releasably securable to said neck flange; and
a tracheostomy tube having a connector with a cuff, said connector insertable into said body flange and said cuff having a lip to engage an edge of said opening to couple said tracheostomy tube to said neck flange;
wherein said tracheostomy tube is attached to a source of oxygen.

2. The tracheostomy support device according to claim 1 wherein said ends each having tie mounting holes for receipt of trach ties, wherein said trach ties secure said neck flange to the patient's neck.

3. The tracheostomy support device according to claim 1 wherein said cuff includes a tab for engaging a notch in the rim of said opening.

4. The tracheostomy support device according to claim 1 wherein said body flange including a swivel connector constructed and arranged to permit 360 rotation of said tracheostomy tube.

5. The tracheostomy support device according to claim 1 wherein said centrally disposed opening is formed from a translucent material, wherein an operator is able to view said body flange during installation.

6. The tracheostomy support device according to claim 1 wherein said tracheostomy tube is coupled to said body flange with pivot pins, said pivot pins allowing said body flange to rotate in relation to said tracheostomy tube.

7. The tracheostomy support device according to claim 1 including an inner cannula having connector tabs for engagement to said connector.

8. The tracheostomy support device according to claim 7 wherein said inner cannula includes a 15 millimeter connector to lock the inner cannula to an outer cannula.

9. The tracheostomy support device according to claim 7 wherein said inner cannula is fenestrated.

10. The tracheostomy support device according to claim 1 wherein said centrally disposed opening includes an offset inlet for the slidable insertion of said cuff.

11. A tracheostomy kit, comprising:
a tracheostomy support device having a neck flange having two ends with a centrally disposed opening therebetween, said neck flange releasable secured to a patients neck; a body flange releasably securable to said neck flange; and a tracheostomy tube having a connector with a cuff, said connector insertable into said body flange and said cuff having a lip to engage an edge of said opening to couple said tracheostomy tube to said neck flange;
gloves for use by a caretaker in handling said kit;
a tray for holding of fluids;
a container for storing hydrogen peroxide, said hydrogen peroxide placed in a first compartment of the tray for used cleaning said tracheostomy support device and neck area of an individual;
at least one cotton swap for use in cleaning said tracheostomy support device and neck area of an individual;
at least one brush for use in cleaning said tracheostomy support device and neck area of an individual;
at least one pipe head for use in cleaning said tracheostomy support device and neck area of an individual;
a container of sterile water, said sterile water for rinsing said tracheostomy neck flange and the neck area of the individual;
a gauze sized for placement between said tracheostomy neck flange and the neck area of the individual; and
a plurality of ties for securing said neck flange to an individual.
